(19) United States
(12) Patent Application Publication
(10) Pub. No.: US 2014/0010806 A1
(43) Pub. Date: Jan. 9, 2014

(54) Treatment of Ocular Diseases

(71) Applicants: James M. Frincke, Del Mar, CA (US); Christopher L. Reading, San Diego, CA (US); Clarence N. Ahlem, San Diego, CA (US)

(72) Inventors: James M. Frincke, Del Mar, CA (US); Christopher L. Reading, San Diego, CA (US); Clarence N. Ahlem, San Diego, CA (US)

(21) Appl. No.: 13/835,995

(22) Filed: Mar. 15, 2013

Related U.S. Application Data
(60) Provisional application No. 61/668,294, filed on Jul. 5, 2012.

(51) Int. Cl. A61K 31/567; A61K 31/573; A61K 9/00; A61K 45/06

(52) U.S. Cl. CPC A61K 31/567; A61K 45/06; A61K 31/573; A61K 9/0048
USPC 424/133.1; 514/182; 514/170

(57) ABSTRACT

The invention relates to methods to use 17α-ethynylandrost-5-ene-3β,7β,17β-triol to treat ocular diseases or conditions such as dry eye, uveitis or retinitis. The compound can be administered topically to the eye, by intravitreal injection or systemically, e.g., orally.

### CROSS REFERENCE TO RELATED APPLICATIONS

This application claims priority to pending U.S. provisional application Ser. No. 61/668,294, filed Jul. 5, 2012, which is incorporated herein by reference.

### FIELD OF THE INVENTION

The invention relates to methods to use 17α-ethynylandrost-5-ene-3β,7β,17β-triol to treat ocular diseases or conditions such as a dry eye condition or uveitis. The compound can be administered locally by injection or topically to the eye, e.g., as sterile solutions or drops.

### BACKGROUND

Therapeutic agents for treating ocular conditions are known, but typically those agents are associated with the development of one or more side-effects. For example, ocular corticosteroid treatment can induce unwanted increases in intraocular pressure or prostaglandin treatments, e.g., PGF_{2α}, can induce hyperemia.

### DESCRIPTION OF THE INVENTION

Summary of invention embodiments. In a principal embodiment, the method provides a method to treat an ocular disease or condition, comprising (or consisting essentially of or consisting of) administering an effective amount of 17α-ethynylandrost-5-ene-3β,7β,17β-triol to a patient in need thereof. The ocular disease or condition to be treated can be dry eye or dry eye syndrome, retinitis, macular degeneration, glaucoma or uveitis.

In some embodiments, 17α-ethynylandrost-5-ene-3β,7β,17β-triol is used to treat or ameliorate inflammation associated with an ocular disease or condition.

Other embodiments are as described elsewhere in the specification including the claims.

Detailed description. As used herein and unless otherwise stated or implied by context, terms that are used herein have the meanings that are defined here. The descriptions of embodiments and examples that are described illustrate the invention and they are not intended to limit it in any way. Unless otherwise contraindicated or implied, e.g., by including mutually exclusive elements or options, in these definitions and throughout this specification, the terms “a” and “an” mean one or more and the term “or” means and/or.

A “patient” means a human.

It has been found that the compound 17α-ethynylandrost-5-ene-3β,7β,17β-triol can be used to treat ocular conditions with limited or minimal unwanted side-effects. Such treatments can be combined with other known treatment agents or drugs with benefit of reducing some side-effects associated with the known drug while not significantly impairing the known drug's efficacy. Other aspects of the invention will become apparent from the description that follows.

Aspects of the activity of 17α-ethynylandrost-5-ene-3β,7β,17β-triol is that it (i) can decrease inflammation by affecting mediators of inflammation such as NF-κB, IL-6 or TNFα and (ii) ameliorate unwanted side-effects of some therapies used to treat ocular diseases or conditions, e.g., dexamethasone or prednisolone. The NF-κB molecule often is an important mediator of inflammation. Increased activation of NF-κB is associated with a range of inflammatory diseases and autoimmune conditions. In addition, 17α-ethynylandrost-5-ene-3β,7β,17β-triol crosses the blood-brain barrier and thus can reach the optic nerve, retina and other ocular structures.

17α-Ethynylandrost-5-ene-3β,7β,17β-triol can be administered systemically, e.g., orally or parenterally, or topically to the eye. For systemic administration, oral administration is preferred. For parenteral administration, intramuscular injection is preferred over intravenous or subcutaneous injection.

The methods described herein are useful to treat, ameliorate or slow the progression of ocular conditions described herein and/or one or more of their symptoms. The patients may be previously or concurrently treated with other conventional treatments, e.g., antibiotics or corticosteroids such as prednisolone, methyl prednisolone or dexamethasone, which may be administered topically or systemically.

Without being limited to any one theory, 17α-ethynylandrost-5-ene-3β,7β,17β-triol would be effective in treating ocular diseases or conditions (or their symptoms) by ameliorating inflammation and/or by increasing healing or repair of injured ocular tissue or cells. Enhanced cell or tissue repair would slow ocular disease progression, enhance recovery or render an existing disease (usually mild to moderate) sub-clinical or nearly sub-clinical.

Treatment with 17α-ethynylandrost-5-ene-3β,7β,17β-triol can thus be continuous or discontinuous, e.g., for as long as the ocular disease is clinically overt. For ocular conditions that can progress over long periods of time, e.g., macular degeneration or an optic nerve disorder, the drug can be administered continuously including during periods when the patient is asymptomatic but still has the underlying disease or condition.

When administered systemically, preferably orally, the patient will receive about 10 mg/day to about 800 mg/day of 17α-ethynylandrost-5-ene-3β,7β,17β-triol, preferably about 20 mg/day to about 100 mg/day. In some embodiments, 20 mg/day to about 400 mg/day of 17α-ethynylandrost-5-ene-3β,7β,17β-triol is administered. In some embodiments, about 40 mg/day to about 120 mg/day or about 200 mg/day of 17α-ethynylandrost-5-ene-3β,7β,17β-triol is administered. The daily doses will be administered once per day or twice per day as subdivided doses, e.g., 100 mg administered once per day or twice as two 50 mg doses.

Solutions or suspensions for topical (ocular) administration containing 17α-ethynylandrost-5-ene-3β,7β,17β-triol will preferably be sterile, isotonic and contain about 0.01% w/w to about 1% w/w of 17α-ethynylandrost-5-ene-3β,7β,17β-triol, preferably about 0.1% w/w to about 0.4% w/w of 17α-ethynylandrost-5-ene-3β,7β,17β-triol, e.g., about 0.2% w/w or 0.3% w/w. Such solutions or suspensions can contain salts, buffers and preservatives typically used in eye drops. Formulations for administration to the eye will preferably limit or omit excipients that can cause eye irritation, e.g., some preservatives such as benzalkonium chloride.

Topical administration to the eye will be one to four times per day, preferably once or twice per day. One to three drops, preferably one or two, will usually be administered to each eye each time eye drops are administered. Drop sizes can vary, but will usually be about 20-60 μL/drop, preferably about 30-50 μL/drop.

Means to dispense topical eye drops have been described and can be used to deliver 17α-ethynylandrost-5-ene-3β,7β,17β-triol to the eye, e.g., as described in U.S. Pat. No. 7,846,140 or other publications.

In addition to administration into patients, 17α-ethynylandrost-5-ene-3β,7β,17β-triol can be administered to animals having, or subject to developing, an ocular disease or condition. In some of these embodiments, the animal will be an animal model suitable for evaluation of the efficacy of topical (ocular) and systemic treatments for various ocular diseases and conditions, e.g., uveitis (Rosenbaum et al, Nature, 286, 611-613, 1980; Rosenbaum et al, Archives of Ophthalmology, 110(4):547-9, 1992), dry eye (Barabino et al., Investigative Ophthalmology & Visual Science, 45(6):1641-1646, 2004; Xiong et al, Investigative Ophthalmology & Visual Science, 49(5):1850-1856, 2008), macular degeneration (Ambati et al, Nature Medicine 9:1390-1397, 2003; Umeda et al, Investigative Ophthalmology & Visual Science, 46(2):683-691, 2005), blepharitis (Mondino et al, Archives of Ophthalmology, 105:409-412, 1987; Sundberg et al, Laboratory Animal Science, 41: 516-518, 1991) and retinopathy, glaucoma and optic neuropathy (Pang, Lok-Hou and Clark, Abbot F. (Eds.), Animal Models for Retinal Diseases, Humana Press, 2010; Pang et al, J. Glaucoma, 16(5):483-505, 2007).

In related embodiments, 17α-ethynylandrost-5-ene-3β,7β,17β-triol is administered to animals having a model condition for an ocular condition, e.g., dry eye or retinopathy, and the efficacy of other known or experimental treatments is compared therewith. In these embodiments, results obtained from treatment of animals having the ocular disease or condition are optionally compared to suitable control animals, e.g., normal controls and/or untreated animals having the ocular disease or condition in addition to animals having the ocular disease or condition that are treated with an experimental drug or therapy.

Formulations suitable for intravitreal injection of will typically be isotonic and sterile and contain 17α-ethynylandrost-5-ene-3β,7β,17β-triol in solution or as a suspension. In one formulation, each mL of the sterile, aqueous formulation provides about 5-40 mg of 17α-ethynylandrost-5-ene-3β,7β,17β-triol. Sodium chloride is used for isotonicity, and about 0.5% (w/v) carboxymethylcellulose sodium, about 0.015% polysorbate 80 and water for injection is also present. The formulation optionally also contains less than about 0.01% (w/v) potassium chloride, calcium chloride (dihydrate), magnesium chloride (hexahydrate), sodium acetate (trihydrate) and/or sodium citrate (dihydrate). Sodium hydroxide and hydrochloric acid may be present to adjust pH to a target value 6-7.5. Other vehicles, e.g., Ringer's solution, can also be used for intravitreal injection formulations.

Variations and modifications of the embodiments, claims and other portions of this disclosure will be apparent to the skilled artisan after a reading thereof. Such variations and modifications are within the scope of this invention. All citations or references cited herein are incorporated herein by reference in their entirety.

The following numbered embodiments further describe the invention or aspects thereof.

1. A method to treat an ocular disease or condition, comprising administering an effective amount of 17α-ethynylandrost-5-ene-3β,7β,17β-triol to a patient in need thereof. In preferred embodiments and for other descriptions herein, the patient in need thereof will have been diagnosed with the ocular disease or condition. In other embodiments, the patient in need thereof will have a condition that precedes the development or clinical manifestation of the ocular disease or condition. In other embodiments, the 17α-ethynylandrost-5-ene-3β,7β,17β-triol is administered orally, as a sterile solution or suspension as topical eye drops or parenterally by intravitreal injection, e.g., in sterile Ringer's solution or other formulations such as isotonic solutions.

2. The method of embodiment 1 wherein the ocular disease or condition is dry eye or dry eye syndrome.

3. The method of embodiment 2 wherein the ocular disease or condition is Sjögren's syndrome.

4. The method of embodiment 2 wherein the ocular disease or condition is keratoconjunctivitis sicca.

5. The method of embodiment 1 wherein the ocular disease or condition is a retinitis condition or retinal disorder.

6. The method of embodiment 5 wherein the retinitis condition or retinal disorder is diabetic retinitis (diabetic retinopathy).

7. The method of embodiment 5 wherein the retinitis condition or retinal disorder is hypertensive retinitis (hypertensive retinopathy).

8. The method of embodiment 5 wherein the ocular disease or condition is macular degeneration.

9. The method of embodiment 8 wherein the macular degeneration is age-related macular degeneration.

10. The method of embodiment 5 wherein the retinitis condition or retinal disorder is retinal detachment.

11. The method of embodiment 5 wherein the retinitis condition or retinal disorder is retinal artery or vein occlusion.

12. The method of embodiment 5 wherein the retinitis condition or retinal disorder is retinal degeneration.

13. The method of embodiment 5 wherein the retinitis condition or retinal disorder is retinitis pigmentosa.

14. The method of embodiment 5, 6, 7, 8, 9, 10, 11, 12 or 13 wherein the patient has been (or is being) treated with (i) an anti-inflammatory agent, optionally prednisolone (e.g., topically), dexamethasone (e.g., topically), bevacizumab (e.g., by intravitreal injection), or (ii) a systemic or topical (ocular) antibiotic.

15. The method of embodiment 1 wherein the ocular disease or condition is elevated intraocular pressure (elevated IOP) (e.g., before or without development of a clinically defined glaucoma) or a glaucoma condition. The treatments will ameliorate elevated IOP or glaucoma symptoms or slow the progression thereof. The glaucoma condition can be a low-tension or normal-tension glaucoma or a glaucoma associated with elevated IOP.

16. The method of embodiment 15 wherein the glaucoma condition is a chronic or idiopathic open-angle glaucoma.

17. The method of embodiment 15 wherein the glaucoma condition is a pupillary block glaucoma.

18. The method of embodiment 17 wherein the pupillary block glaucoma is acute angle-closure glaucoma, chronic angle-closure glaucoma or combined mechanism glaucoma.

19. The method of embodiment 15, 16, 17 or 18 wherein the patient has hypertension or diabetes. The ocular condition can be diabetic macular edema.

20. The method of embodiment 15, 16, 17 or 18 wherein the patient has been (or is being) treated with corticosteroids, optionally topical prednisolone.

21. The method of embodiment 15, 16, 17 or 18 wherein the patient has been (or is being) treated with a cholinergic agonist, optionally pilocarpine or carbachol.

22. The method of embodiment 15, 16, 17 or 18 wherein the patient has been (or is being) treated with a topical β-blocker, optionally timolol, betaxolol or levobunolol.

23. The method of embodiment 15, 16, 17 or 18 wherein the patient has been (or is being) treated with a topical prostaglandin or prostaglandin analog, optionally latanoprost.

24. The method of embodiment 15, 16, 17 or 18 wherein the patient has been (or is being) treated with a carbonic anhydrase inhibitor, optionally oral or IV acetazolamide or oral dichlorphenamide.

25. The method of embodiment 1 wherein the ocular disease or condition is uveitis.

26. The method of embodiment 1 wherein the ocular disease or condition is an optic nerve disease or disorder.

27. The method of embodiment 26 wherein the optic nerve disease or disorder is papilledema.

28. The method of embodiment 26 wherein the optic nerve disease or disorder is optic neuritis.

29. The method of embodiment 26 wherein the optic nerve disease or disorder is retrobulbar neuritis.

30. The method of embodiment 1 wherein the ocular disease or condition is ocular inflammation or discomfort or trauma caused by or associated with the use of contact lenses.

31. The method of embodiment 1 wherein the ocular disease or condition is ocular inflammation, discomfort or trauma caused by or associated with refractive surgery, optionally radial keratotomy or astigmatic keratotomy.

32. The method of embodiment 1 wherein the ocular disease or condition is blepharitis.

33. The method of embodiment 30, 31 or 32 wherein the patient has been (or is being) treated with topical corticosteroids, optionally prednisolone or dexamethasone.

34. The method of embodiment 1 wherein the ocular disease or condition is a conjunctivitis condition.

35. The method of embodiment 34 wherein the conjunctivitis condition is allergic conjunctivitis.

36. The method of embodiment 34 wherein the conjunctivitis condition is pink eye.

37. The method of embodiment 34 wherein the conjunctivitis condition is giant papillary conjunctivitis.

38. The method of embodiment 34 wherein the conjunctivitis condition is infectious conjunctivitis (bacterial, viral or chlamydial).

39. The method of embodiment 34 wherein the conjunctivitis condition is chemical conjunctivitis, optionally chlorine- or air pollution-induced.

40. The method of any preceding embodiment, e.g., embodiment 1, 2, 3, 4, 14, 25, 30, 31, 32, 33, 34, 35, 36, 37, 38 or 39 wherein the 17α-ethynylandrost-5-ene-3β,7β,17β-triol is administered topically to the eye, optionally as sterile eye drops or by intravitreal injection of a sterile solution or, less preferably, a sterile suspension.

41. The method of any preceding embodiment, e.g., embodiment 1, 5, 6, 7, 8, 9, 10, 11, 12, 14, 15, 19, 20, 25, 26, 32, 34 or 35, wherein the 17α-ethynylandrost-5-ene-3β,7β,17β-triol is administered systemically, preferably orally.

42. The method of embodiment 41 wherein the ocular disease or condition is uveitis and the 17α-ethynylandrost-5-ene-3β,7β,17β-triol is administered orally.

43. The method of embodiment 41 wherein the ocular disease or condition is retinopathy and the 17α-ethynylandrost-5-ene-3β,7β,17β-triol is administered orally.

44. The method of embodiment 41 wherein the ocular disease or condition is macular degeneration and the 17α-ethynylandrost-5-ene-3β,7β,17β-triol is administered orally.

45. The method of embodiment 41, 42, 43 or 44 wherein about 20 mg/day to about 400 mg/day of 17α-ethynylandrost-5-ene-3β,7β,17β-triol is administered.

46. The method of embodiment 41, 42, 43 or 44 wherein about 40 mg/day to about 120 mg/day of 17α-ethynylandrost-5-ene-3β,7β,17β-triol is administered, optionally about 80 mg/day.

47. Use of 17α-ethynylandrost-5-ene-3β,7β,17β-triol in the treatment or prophylaxis of an ocular disease or condition.

48. Use according to embodiment 47 wherein the ocular disease or condition is a ocular disease or condition as recited in any of embodiments 2-32.

49. Use according to embodiment 47 or 48 wherein a second therapeutic agent, optionally a corticosteroid such as prednisolone or dexamethasone, is also used in the treatment or prophylaxis of the ocular disease or condition.

50. Use according to embodiment 47, 48 or 49 wherein the 17α-ethynylandrost-5-ene-3β,7β,17β-triol is in a formulation for administration topically to the eye, optionally as sterile eye drops.

51. Use according to embodiment 47, 48 or 49 wherein the 17α-ethynylandrost-5-ene-3β,7β,17β-triol is in a formulation for systemic administration, optionally a formulation for oral administration.

52. Use according to embodiment 51 wherein the formulation contains about 20 mg/day to about 400 mg/day of 17α-ethynylandrost-5-ene-3β,7β,17β-triol.

53. Use according to embodiment 51 wherein the formulation contains about 40 mg/day to about 120 mg/day of 17α-ethynylandrost-5-ene-3β,7β,17β-triol, optionally about 80 mg/day.

54. A method to treat an ocular disease or condition in a non-human animal or a non-human animal having a model disease for the corresponding human ocular disease or condition, comprising (or consisting essentially of or consisting of) (a) administering an effective amount of 17α-ethynylandrost-5-ene-3β,7β,17β-triol to the non-human animal having the ocular disease or condition, or an animal model for the corresponding human disease, (b) assessing the effect of the treatment of step (a), and optionally (c) treating one or more additional non-human animals having the ocular disease or condition with (i) placebo, (ii) an experimental drug or therapy and/or (iii) a drug or therapy that has been used to treat the ocular disease or condition or the corresponding human disease and comparing the results or effects of treatment(s) in step (c) with the assessed effect of step (b). In this embodiment, the

55. The method of embodiment 54 wherein the ocular disease or condition is (i) dry eye or dry eye syndrome, (ii) a retinitis condition or retinal disorder, (iii) macular degeneration, (iv) a glaucoma condition, (v) uveitis, (vi) dry eye, (vii) conjunctivitis, or (viii) pink eye.

56. The method of embodiment 54 or 55 wherein the non-human animal is a mouse, rat, rabbit or monkey.

### EXAMPLES

The following examples further illustrate the invention and they are not intended to limit it in any way.

### Example 1

Reduction of inflammation. Inhibition of NF-κB by 17α-ethynylandrost-5-ene-3β,7β,17β-triol in vitro. A number of compounds were used to inhibit activation of NF-κB by TNF-α or LPS in human cells in vitro. Activation of NF-κB increases expression of a number of genes that mediate inflammation. This protocol used human THP-1 cells, which are human mononuclear blood cells with a monocyte phenotype. The cell line, referred to as NF-κB-bla THP-1, contained a β-lactamase reporter gene under the control of the NF-κB response element (Invitrogen, CellSensor™, product No. K1176). In this cell line, the β-lactamase reporter gene is stably integrated in the THP-1 cells. This cell line was used to detect agonists or antagonists of the NF-κB signaling pathway. These NF-κB-bla THP-1 cells respond to the presence of tumor necrosis factor alpha (TNFα) or bacterial lipopolysaccharide (LPS) by increased expression of the β-lactamase reporter gene. The level of β-lactamase enzyme activity was measured by fluorescence resonance energy transfer ratiometric detection. TNFα and LPS are both potent inflammation-inducing agents that activate NF-κB in THP1 cells. In this assay, compounds that decrease NF-κB activity, and thus β-lactamase, in the presence of TNFα or LPS are exerting an anti-inflammation activity.

The NF-κB-bla THP-1 cells were maintained by passaging or feeding as needed. The cells, which grow in suspension, were maintained at a density between 2×10⁵ cells per mL and 2×10⁶ cells/mL. The cells were plated at 20,000 cells/well in a 384-well Black-wall, clear bottom assay plates (Costar #3712-TC low fluorescence background plates) approximately 24 hours before adding either TNFα at 10 ng/mL or LPS at 0.2 ng/mL to activate NF-κB. In positive control assays for activation of NF-κB, the EC₅₀ concentration for TNFα was 0.20 ng/mL after a 1 hour β-lactamase substrate incubation. The EC₅₀ dose for LPS was 0.15 ng/mL. The EC₅₀ concentration for TNF-α or LPS in this assay refers to 50% of the concentration of TNF-α or LPS that causes a maximum activation of NF-κB. The synthetic glucocorticoid dexamethasone (a potent anti-inflammatory drug) decreased the effect of TNFα by with an EC₅₀ of 0.47 nM (average of 5 assays) in this assay. Similar biological activity for dexamethasone has been reported in other in vitro cell assays, with complete inhibition of NF-κB activation observed at an IC₅₀ of about 1 nM (M. K. A. Bauer et al., Eur. J. Biochem. 243:726-731, 1977).

The IC₅₀ concentration for the compounds used in this assay refers to the concentration of compound that causes a 50% of the maximum inhibition of NF-κB activation that the compound can induce. The assays were usually conducted 2-4 times for each compound and the values are averages for each compound. The compound 17α-ethynylandrost-5-ene-3β,7β,17β-triol had an IC₅₀ of 19 fM±11. The IC₅₀ for dexamethasone was 0.47 nM±11, the IC₅₀ for 3β,7β,16α,17β-tetrahydroxyandrost-5-ene was 8.2 fM±7.4, the IC₅₀ for 3α,7β,16α,17β-tetrahydroxyandrost-5-ene was 84.5 fM±65 and the IC₅₀ for 3β,17β-dihydroxy-7-oxo-17α-ethynylandrost-5-ene was 11.5 fM±3.5.

The maximum inhibition of NF-κB by dexamethasone, 16α-bromoepiandrosterone and 16β-bromoepiandrosterone was 100% and there was no detectable NF-κB activation at concentrations of these compounds above the IC₅₀ for these compounds. By contrast, maximum inhibition of NF-κB by the other compounds e.g., 3β,7β,16α,17β-tetrahydroxyandrost-5-ene or 3α,7β,16α,17β-tetrahydroxyandrost-5-ene was less than about 80%, with increasing amounts of the compounds above their IC₅₀ levels not providing significant additional inhibitory activity against NK-κB activation.

### Example 2

The capacity of selected compounds to treat LPS induced inflammation in mice was examined by a protocol similar to the protocol described above. Five groups of three ICR mice weighing about 30 g were each treated by intraperitoneal injection with 120 μL vehicle (30% sulfobutylether-cyclodextrin in water), androst-5-ene-3α,7β,16α,17β-tetrol in vehicle, androst-5-ene-3β,4β,16α,17β-tetrol in vehicle or 4β-acetoxyandrost-5-ene-3β,16α,17β-triol in vehicle. All drug and vehicle formulations were solutions, not suspensions. The sulfobutylether-cyclodextrin was obtained commercially (Cydex Pharmaceuticals, Inc., Lexena, Kans.). There were two vehicle control groups one group received vehicle alone and the other received vehicle plus LPS. The vehicle or drug was administered 24 hours before and at 1 hour after LPS (about an LD_{50/24} dose, i.e., 50% lethal at 24 hours after LPS administration) was administered to the mice by intraperitoneal injection. Drug was administered at about 40 mg/kg (1.2 mg drug/animal for each administration of the drugs). Spleens were harvested from the animals at 1.5 hours after injection of LPS and spleen cells were lysed and assayed for activated NF-κB by isolating nuclei from spleen cells and measuring NF-κB from the lysed nuclei. The results indicated that all three compounds decreased the level of NF-κB activation compared to the LPS+vehicle control group by about 50%. The level of activated NF-κB in spleen cells from the animals that were treated with vehicle and no LPS, was essentially the same as the activated NF-κB in spleen cells from drug treated animals. These results indicated a potent anti-inflammation effect in the animals as shown by a decrease in activated NF-κB in drug treated animals compared to control animals.

### Example 3

Kinetic analysis of NF-kB inhibition in vivo. The kinetics of NF-kB inhibition after injection of bacterial LPS in mice was examined to further probe the mechanism of action of compounds such as 17α-ethynylandrost-5-ene-3β,7β,17β-triol, which will only partially inhibit activation of NF-κB that is induced by LPS or TNFα in immune cells (macrophages or monocytes) in vitro. In this study, mice were treated with 17α-ethynylandrost-5-ene-3β,7β,17β-triol (about 40 mg/kg, about 1.2 mg/animal) by intraperitoneal injection of a solution (not a suspension) of the compound in vehicle (120 μL vehicle; 30% sulfobutylether-cyclodextrin in water). The drug was injected 24 hours before intraperitoneal injection of bacterial LPS (about an LD_{50/24}). The study used two groups of 12 animals, vehicle control or drug administered 24 hours before LPS challenge. Spleens were harvested from 3 animals from both groups just before LPS challenge and at 1.5, 2.0 and 2.5 hours after administration of LPS. Spleen cells were harvested and the level of activated NF-κB was measured by assay of NF-κB in nuclei.

Maximum NF-κB activation after LPS administration occurred at 1.5 hours in the vehicle controls, which was 4-fold increased over the pre-LPS level of activated NF-κB. The results are shown below. The values for the vehicle control and drug treated animals are relative optical density units from ELISA measurement of NF-κB in nuclei from spleen cells.

| Time | vehicle | drug |
|---|---|---|
| (hours) | control | treated |
| 0 | 18 | 22 |
| 1.5 | 72 | 2 |
| 2.0 | 10 | 7 |
| 2.5 | 10 | 9 |

The profound inhibition of NF-κB at the 1.5 hour time point and relatively normal levels of NF-κB activity at the other time points indicated that the compound exerted a transient but potent inhibition of LPS induced trauma at a critical period after LPS exposure. Similar assays in other studies showed that the level of activated NF-κB at 30 minutes and 60 minutes after injection of LPS in vehicle control mice was similar to the pre-LPS time point in this study. This result indicates that in this model, the effect of LPS on the activation of NF-κB in spleen cells is maximal at about 1.5 hours post LPS challenge. This time point reveals a convenient time or window at which the activity of anti-inflammatory drug candidates can be assessed in vivo, i.e., at about 75 minutes to about 105 minutes after LPS challenge. The window can vary, depending on the route of administration of the biological insult, e.g., LPS or TNFα, administered by intraperitoneal injection versus LPS or TNFα administered by subcutaneous or intramuscular injection.

Analysis of LPS induced TNFα expression in mice showed that TNFα levels peaked at 1.5 hours after LPS challenge (500 μg of LPS administered by intraperitoneal injection) with highest levels of TNFα observed at 1-2 hours after LPS challenge. TNFα levels at 30 minutes after LPS and at 2.5 hours were lower.

### Example 4

Analysis of immune suppression. Glucocorticoid steroids such as dexamethasone, prednisolone or hydrocortisone are typically immune suppressive and have significant toxicities associated with their use. Immune suppression was examined in a reporter antigen popliteal lymph node assay in mice essentially as previously described (C. Goebel et al., Inflamm. Res., 45(Suppl. 2):S85-S90, 1996; R. Pieters et al., Environmental Health Perspectives 107(Suppl. 5):673-677, 1999). This protocol was used to analyze the activity of 17α-ethynylandrost-5-ene-3β,7β,17β-triol in the popliteal lymph node (PLN) assay to show that the compound does not have appreciable immune suppression activity in vivo. In this protocol, the vehicle was 0.1% carboxymethylcellulose, 0.9% saline, 2% tween 80 and 0.05% phenol, which contained 17α-ethynylandrost-5-ene-3β,7β,17β-triol in suspension in drug treated animals. Assessment of activity included (1) measuring suppression of numbers of total lymphocytes, antigen specific IgM, IgG1 and IgG2a antibody secreting cells (ASC) (ELISPOT assay) in popliteal lymph node cells; (2) analysis of cell surface marker (CD4, CD8, CD19, F480, CD80, CD86) expression by flow cytometry of living cells in suspension; and (3) IL-4, TNFα and IFNγ production by lymphocytes in vitro (ELISA).

Groups (n=5 per group) of specific pathogen free BALB/C mice were used. The Positive control group was treated with vehicle (oral gavage) and 5 μg/day dexamethasone by subcutaneous injection to induce immune suppression. Vehicle control animals (negative control) were treated with vehicle alone (oral gavage). One group of animals was treated with 17α-ethynylandrost-5-ene-3β,7β,17β-triol at 0.1 mg/day by oral gavage. Another group was treated with 1 mg/day of 17α-ethynylandrost-5-ene-3β,7β,17β-triol was administered to the animals by oral gavage. The results were analyzed by two-tailed Student's t-test with equal variance. The animals were injected in the right hind footpad with 50 μL of freshly prepared sensitizing dose of TNP-OVA. Dexamethasone (decadron phosphate injection; dexamethasone sodium phosphate) was administered by subcutaneous injection into the nape of the neck daily, immediately following sensitization with TNP-OVA. 17α-Ethynylandrost-5-ene-3β,7β,17β-triol was given immediately afterwards by gavage. Five days after injection of TNP-OVA, blood was drawn by orbital puncture, and the mice were euthanized by cervical dislocation and popliteal lymph nodes were removed and separated from adherent fatty tissue. Single cell suspensions were prepared, resuspended in 1 mL PBS-BSA (1%) and counted. Cell numbers, IL-4, IL-5 and IFNγ were measured.

The average number of lymphocytes in PLNs from the vehicle control group was 7.8×10⁶ per lymph node compared to 2.9×10⁶ per lymph node in the dexamethasone treated animal group. This reduced lymphocyte count clearly showed the marked immune suppression that is typically seen with the use of dexamethasone or other glucocorticoid compounds. By contrast, the group treated with 1 mg/day of 17α-ethynylandrost-5-ene-3β,7β,17β-triol had 8.2×10⁶ lymphocytes per lymph node and the group treated with 0.1 mg/day of 17α-ethynylandrost-5-ene-3β,7β,17β-triol had 11.1×10⁶ lymphocytes per lymph node. The results showed that 17α-ethynylandrost-5-ene-3β,7β,17β-triol was not immune suppressive, but was immune enhancing. 17α-Ethynylandrost-5-ene-3β,7β,17β-triol treatment at 1.0 mg/day and at 0.1 mg/day increased IFNγ, IL-4 and IL-5 levels compared to the vehicle control group, also indicating immune enhancement. The effect of 17α-ethynylandrost-5-ene-3β,7β,17β-triol at 0.1 mg/day on IFNγ, IL-4 and IL-5 levels was greater than in the group that was treated with 1.0 mg/day. By contrast, IFNγ, IL-4 and IL-5 levels were reduced in the dexamethasone treated group compared to the vehicle control group or to either drug treated group.

### Example 5

Analysis of immune suppression. Several compounds were characterized for their capacity to affect immune responses. This protocol examined the immune effects of compounds in a standard immune assay. The ovalbumin (OVA) specific immune response assay is a well-established system to measure anamnestic (both cell-mediated and antibody-mediated) immune responses. BALB/c mice were immunized by intraperitoneal injection (total volume 200 μL) on days 0 and 7 with 100 μg OVA precipitated with alum (25 mg/mL) in saline. Mice (n=5 per group) were treated daily (oral gavage 40 mg/kg, about 1 mg/animal) for 20 days with compound. On day 20, blood was drawn and tested in ELISA for antibody titers to OVA. The compounds that were tested were 3β,16α-dihydroxy-17-oxoandrostane, 16α-bromoepiandrosterone, 17α-ethynylandrost-5-ene-3β,7β,17β-triol, 3β,16α-dihydroxyandrostane-17-oxime, 17β-aminoandrost-5-ene-3β-ol and 3α,16α,17β-trihydroxyandrostane. None of these compounds were profoundly immune suppressive, with OVA antibody titers similar to those in the vehicle control group.

### Example 6

The capacity of compounds including 17β-ethynylandrost-5-ene-3β,7β,17α-triol to treat multiple sclerosis (MS) was evaluated in experimental autoimmune encephalomyelitis (EAE). The protocol for conducting the EAE animal model was described in (D. Auci et. al., Ann. NY. Acad. Sci. USA, 1051:730-42, 2005). Female SJL mice (6-8 week old, average body weight of 25 g) obtained from Charles-River were kept under standard laboratory conditions (non specific pathogen germ free) with ad libitum food and water and were allowed to adapt one week to their environment before commencing the study. Animals were randomized into six groups of seven animals each and were (1) mice treated with vehicle, (2) mice treated with SU5416 (Z-3-[(2,4-dimethylpyrrol-5-yl)methylidenyl]-2-indolinone), (3) mice treated with 17β-ethynylandrost-5-ene-3β,7β,17α-triol, (4) mice treated with androst-5-ene-3α,7β,16α,17β-tetrol, (5) mice treated with androst-5-ene-3β,7β,16α,17β-tetrol, (6) mice treated with 3α-trifluoromethylandrost-5-ene-3β,17β-diol, (7) mice treated with 17α-trifluoromethyl-androst-5-ene-3β,17β-diol and (8) mice treated with 5α-androstane-3β,17β-diol-16-oxime. EAA was induced with 200 μL of a 1:1 emulsion of 75 μg proteolipid protein (PLP) and 6 mg/mL Mycobacterium tuberculosis H37RA in complete Freund's adjuvant (CFA). The 200 μL injection was divided among four sites that drained into the axillary and inguinal lymph nodes. Pertussis toxin was used as a co-adjuvant and was administered i.p. at 200 ng/mouse on day zero and day two post immunization. Groups were treated with 0.1 mg of compound in 100 μL vehicle, or with vehicle alone, q.d. po (oral gavage) starting at clinical onset of disease and continuing through to day 30 post immunization. Clinical onset is defined as the time when clinical symptoms of the disease attain a grading between 2-3 in 25% of the mice. Clinical grading was carried out by an observer unaware of the treatment: 0=no illness, 1=flaccid tail, 2=moderate paraparesis, 3=severe paraparesis, 4=moribund state, 5=death. Statistical analysis for significant differences on clinical scores was performed by ANOVA for unpaired data and to the non parametric Mann-Whitney test. A P value <0.05 was considered to be statistically significant. For statistical analysis, the mice that succumbed to EAE were assigned 5 only for the day of death and then were deleted from the experimental group.

As expected, classical signs of EAE developed in 8/8 (100%) of the vehicle-treated mice within day 19^{th} post immunization. The mean day of onset was 15.5±3.9 (SD). In this group of animals the duration of the disease was 12.3±4.3 days. The mean cumulative score from day 1 to 30 was 24.8±7.8 and that from day 31 to day 54 (post treatment) was 22.7±15.8. A course of EAE very similar to that observed in the vehicle-treated mice was observed in the animals treated with SU5416, androst-5-ene-3α,7β,16α,17β-tetrol and 5α-androstane-3β,17β-diol-16-oxime, the so-treated mice exhibiting cumulative incidence of disease, duration of disease and mean cumulative onset comparable to that of the controls. In contrast, the mice treated with androst-5-ene-3β,7β,16α,17β-tetrol, 3α-trifluoromethylandrost-5-ene-3β,17β-diol or 17α-trifluoromethylandrost-5-ene-3β,17β-diol exhibited a significantly improved course of EAE as compared to the vehicle-treated mice entailing significantly reduction of both one or more the mean cumulative score and duration. And in further contrast, none of these 3 compounds significantly influenced the cumulative incidence of EAE or the lethality. Finally, although 17β-ethynylandrost-5-ene-3β,7β,17α-triol only exhibited a trend toward reduced cumulative score and duration vs. the vehicle-treated mice, the effects appeared to be biological important (14.9±17.6 and 7±7.9 vs. 24.8±7.8 and 12.3±4.3). The lack of statistical significance with this compound is probably due to the large number of mice being assigned score 0 throughout the observation period which therefore resulted in a high standard deviation.

At the end of the treatment on day 30^{th}, the mice were monitored for up to additional 24 days. It was possible to observe the disease becoming chronic in the vehicle-treated mice with cumulative scores comparable to that of the treatment period. A substantial increase in the cumulative score during the follow-up period as compared to the treatment period was observed with SU5416 that passed from a mean cumulative score of 25.5±8.9 to 35.5±13.2 and more modestly with 17β-ethynylandrost-5-ene-3β,7β,17α-triol that passed from a mean cumulative score of 14.9±17.6 to 18.4±20.6. In the mice treated with 17β-ethynylandrost-5-ene-3β,7β,17α-triol t it was also possible to observe an increase of the EAE incidence from 57.1% at the end of the treatment period to 85.7% at the end of the follow-up period. On the other hand, the other compounds have appeared to maintain a similar cumulative score in the follow-up period as in the treatment period. This was particularly remarkable for 3α-trifluoromethylandrost-5-ene-3β,17β-diol that passed from a mean cumulative score of 11.2±4.8 during the treatment period to 10.8±10.3 at the end of the follow-up period.

These results show that 17β-ethynylandrost-5-ene-3β,7β,17α-triol, androst-5-ene-3β,7β,16α,17β-tetrol, 3α-trifluoromethylandrost-5-ene-3β,17β-diol and 17α-trifluoromethyl-androst-5-ene-3β,17β-diol exerted powerful anti-inflammatory properties in the PLP-induced model of EAE in SJL mice. Of particular relevance for the translation of these findings to the clinical setting are the observations that the compounds are active in this EAE model even when given in a protocol starting on day 12^{th} post immunization when 24% of the mice had already developed clinical signs of EAE. Of particular note is the finding that SU5416 was ineffective in this setting. It has been previously reported that SU5416 is effective in EAE (L. Bouerat et al., J. Med. Chem. 48: 5412-5414, 2005). However, to obtain this result, the SU5416 compound was administered at the same time the animals were immunized. By contrast, in this protocol compounds such as 17β-ethynylandrost-5-ene-3β,7β,17α-triol were not administered to the animals until after disease symptoms were apparent, which shows that the compounds can be used to effectively treat existing disease and to prevent or delay disease onset.

### Example 7

Treatment of neuron loss associated with trauma and bone loss conditions. Immune competence is a complex function that can be acutely impaired following elevations in endogenous glucocorticoid (GC) levels.

The capacity of 17α-ethynyl-5-androstene-3β,7β,17β-triol to reverse adverse effects of glucocorticoids in bone growth was shown in the human MG-63 osteosarcoma cell line. MG-63 cells are osteoblasts, which are cells that mediate bone growth. This cell line has been used extensively to study bone biology and to characterize the biological activity of compounds for treatment of bone loss conditions (e.g., B. D. Boyan et al., J. Biol. Chem., 264(20):11879-11886, 1989; L. C. Hofbauer et al., Endocrinology, 140(10):4382-4389, 1999). Adverse toxicities associated with elevated glucocorticoid levels include a decrease in the production of IL-6 and IL-8 by osteoblasts, including the MG-63 cell line, and an increase in the expression of the 11β-hydroxysteroid dehydrogenase type 1 enzyme (11β-HSD). Increased 11β-hydroxysteroid dehydrogenase type 1 enzyme results in increased levels of endogenous glucocorticoid activity by converting endogenous cortisone to the active cortisol, which inhibits bone growth. The 11β-HSD enzyme is expressed in liver, adipose tissue, brain and bone tissues. Cortisol generated by 11β-HSD-1 contributes to osteoporosis and disorders such as stroke, neuron death, depression and Parkinson Disease. Decreases in IL-6, IL-8 and osteoprotegerin are associated with decreased bone growth by osteoblasts. Pilot studies showed that the IC₅₀ for inhibition of IL-6 from MG-63 cells by dexamethasone was 10 nM and the IC₅₀ for inhibition of growth of MG-63 cells by dexamethasone was 15.3 nM.

In this protocol, MG-63 cells were grown in the presence or absence of the synthetic glucocorticoid dexamethasone at a 30 nM concentration and in the presence or absence of compound at 10 nM. Compound 1 in the table below was 5-androstene-3β,7β,17β-triol, compound 2 was 17α-ethynylandrost-5-ene-3β,7β,17β-triol and compound 3 was 4-estrene-3α,17β-diol. The results for these compounds are shown below.

| MG-63 growth | IL-6 | IL-8 | 11β-HSD mRNA | osteoprotegerin |
|---|---|---|---|---|
| conditions | pg/mL | units | units | pmol/L |
| vehicle control | 6.2 | 0.90 | 0.25 | 445 |
| dexamethasone | 1.3 | 0.12 | 1.0 | 280 |
| compound 1 | 4.0 | 0.53 | 0.73 | — |
| compound 2 | 2.8 | 0.50 | 0.54 | — |
| compound 2 (1 nM) | — | — | — | 455 |
| compound 3 | 4.1 | 0.55 | 0.75 | — |

These results showed that the compounds at 10 nM partially reversed the adverse effects of dexamethasone at 30 nM, which shows that the compounds can reverse multiple toxicities associated with elevated glucocorticoid levels in osteoblasts, which are the cells that mediate bone growth. In a related protocol, the compound 17α-ethynyl-5-androstene-3β,7β,17β-triol at 1 nM also completely reversed the decrease in osteoprotegerin synthesis by MG-63 cells after growth of the cells for 7 hours in the presence of 30 nM dexamethasone as shown in the table above. Other compounds that completely or partially reversed the decrease in osteoprotegerin synthesis by MG-63 cells in the presence of 30 nM dexamethasone were 5-androstene-3β,7β,16α,17β-triol (normal osteoprotegerin levels at 0.1 μM), 3β,7α,16α,17β-tetrahydroxyandrost-5-ene (near normal osteoprotegerin levels at 10 nM) and 3α,7β,16α,17β-tetrahydroxyandrost-5-ene (normal osteoprotegerin levels at 10 nM).

To show that relevant effects could be obtained in vivo, the compound 17α-ethynyl-5-androstene-3β,7β,17β-triol was administered to mice that were also treated daily with dexamethasone for 23 days to reduce levels of osteoprotegerin in the animals. Osteoprotegerin levels in mice that were treated with vehicle and dexamethasone at 10 μg/day (positive control group) had 3.3 pmol/L osteoprotegerin, while animals treated with vehicle, dexamethasone and 17α-ethynyl-5-androstene-3β,7β,17β-triol at 4 mg/kg/day had 6.4 pmol/L osteoprotegerin (p<0.05). These results showed amelioration of unwanted side effects of corticosteroids such as dexamethasone by 17α-ethynyl-5-androstene-3β,7β,17β-triol.

### Example 8

Eye drop formulations. An exemplary formulation consists essentially of sterile isotonic solution containing approximately 1-3 mg/mL 17α-ethynyl-5-androstene-3β,7β,17β-triol, 3-5% 2-hydroxy-propyl-β-cyclodextrin, phosphate buffered saline, pH 7.4. The solution is packaged in single use ampoules (e.g., blow-fill-seal) or in a multi-dose container if 0.01% benzalkonium chloride is present as a preservative. Methods and apparatus to make sterile blow-fill-seal ampoules have been described, e.g., U.S. Pat. Nos. 5,624,057 and 7,833,195.

Another formulation is a sterile isotonic suspension containing 1-3 mg/mL 17α-ethynyl-5-androstene-3β,7β,17β-triol, 0.1-0.5% of a viscosity imparting agent such as methylcellulose, hydroxypropyl methylcellulose or polyvinyl alcohol, phosphate buffered pH 7.4 and 0.01% benzalkonium chloride. The suspension can be packaged in a multi-dose container. The formulation without benzalkonium chloride can be used in single use containers.

What is claimed is:

1. A method to treat an ocular disease or condition, comprising administering an effective amount of 17α-ethynylandrost-5-ene-3β,7β,17β-triol to a patient in need thereof, wherein the 17α-ethynylandrost-5-ene-3β,7β,17β-triol is administered topically as a sterile eye drop formulation or as a sterile solution by intravitreal injection.

2. The method of claim 1 wherein the ocular disease or condition is dry eye or dry eye syndrome, Sjögren's syndrome, keratoconjunctivitis sicca or a retinitis condition or retinal disorder,

3. The method of claim 2 wherein the patient is further treated with an effective amount of (i) an anti-inflammatory agent, optionally prednisolone, dexamethasone, bevacizumab, or (ii) a systemic or topical antibiotic.

4. The method of claim 2 wherein the retinitis condition or retinal disorder is diabetic retinitis, hypertensive retinitis, retinal detachment, retinal artery or vein occlusion, retinal degeneration, retinitis pigmentosa or macular degeneration, optionally age-related macular degeneration,

5. The method of claim 4 wherein the patient is further treated with an effective amount of (i) an anti-inflammatory agent, optionally prednisolone, dexamethasone, bevacizumab, or (ii) a systemic or topical antibiotic.

6. The method of claim 1 wherein the ocular disease or condition is elevated intraocular pressure or a glaucoma condition.

7. The method of claim 6 wherein the glaucoma condition is a chronic or idiopathic open-angle glaucoma, a pupillary block glaucoma.

8. The method of claim 7 wherein the pupillary block glaucoma is acute angle-closure glaucoma, chronic angle-closure glaucoma or combined mechanism glaucoma.

9. The method of claim 1 wherein the patient has hypertension or diabetes.

10. The method of claim 2 wherein the patient has hypertension or diabetes.

11. The method of claim 6 wherein the patient has hypertension or diabetes.

12. The method of claim 6 wherein the patient is further treated with (i) one or more corticosteroids, optionally topical prednisolone, (ii) a cholinergic agonist, optionally pilocarpine or carbachol, (iii) a topical β-blocker, optionally timolol, betaxolol or levobunolol, (iv) a topical prostaglandin or prostaglandin analog, optionally latanoprost or (v) a carbonic anhydrase inhibitor, optionally acetazolamide or dichlorphenamide.

13. The method of claim 1 wherein the ocular disease or condition is uveitis, optic neuritis, retrobulbar neuritis, ocular inflammation or discomfort or trauma caused by or associated with the use of contact lenses, ocular inflammation, discomfort or trauma caused by or associated with refractive surgery, optionally radial keratotomy or astigmatic keratotomy, blepharitis, an optic nerve disease or disorder, optionally papilledema or a conjunctivitis condition, optionally allergic conjunctivitis, pink eye, giant papillary conjunctivitis, infectious conjunctivitis or chemical conjunctivitis.

14. The method of claim 13 wherein the patient is further treated with topical corticosteroids, optionally prednisolone or dexamethasone.

15. The method of claim 1 wherein the 17α-ethynylandrost-5-ene-3β,7β,17β-triol is administered topically to the eye, optionally as sterile eye drops.

16. The method of claim 1 wherein the 17α-ethynylandrost-5-ene-3β,7β,17β-triol is administered systemically, optionally wherein the ocular disease or condition is uveitis, a retinopathy or macular degeneration.

17. A method to treat an ocular disease or condition in a non-human animal that has the ocular disease or condition or a non-human animal having a model disease for the corresponding human ocular disease or condition, comprising
(a) administering an effective amount of 17α-ethynylandrost-5-ene-3β,7β,17β-triol to the non-human animal having the ocular disease or condition, or an animal model for the corresponding human disease,
(b) assessing the effect of the treatment of step (a), and
(c) treating one or more additional non-human animals having the ocular disease or condition with (i) placebo, (ii) an experimental drug or therapy and/or (iii) a drug or therapy that has been used to treat the ocular disease or condition or the corresponding human disease and comparing the results or effects of treatment(s) in step (c) with the assessed effect of step (b).

18. The method of claim 17 wherein the ocular disease or condition or the model disease for the corresponding human ocular disease or condition is (i) dry eye or dry eye syndrome, (ii) a retinitis condition or retinal disorder, (iii) macular degeneration, (iv) a glaucoma condition, (v) uveitis, (vi) dry eye, (vii) conjunctivitis, or (viii) pink eye.

19. The method of claim 17 wherein the non-human animal is a mouse, rat, rabbit or monkey.
